(19) United States
(12) Patent Application Publication
(10) Pub. No.: US 2025/0033054 A1
(43) Pub. Date: Jan. 30, 2025

(54) MICROFLUIDIC SYSTEMS AND METHODS OF USE

(71) Applicant: 10x Genomics, Inc., Pleasanton, CA (US)

(72) Inventors: Rajiv Bharadwaj, Pleasanton, CA (US); Kevin Ness, Pleasanton, CA (US); Tobias Daniel Wheeler, Pleasanton, CA (US)

(21) Appl. No.: 18/913,548

(22) Filed: Oct. 11, 2024

Related U.S. Application Data
(62) Division of application No. 17/397,146, filed on Aug. 9, 2021, now Pat. No. 12,138,628.
Application No. 17/397,146 is a continuation of application No. 16/180,356, filed on Nov. 5, 2018, now Pat. No. 11,084,036.
Application No. 16/180,356 is a continuation of application No. PCT/US2017/032520, filed on May 12, 2017.
(60) Provisional application No. 62/335,870, filed on May 13, 2016.

(51) Int. Cl. B01L 3/00; G01N 1/28; G01N 33/487

(52) U.S. Cl. CPC B01L 3/502776; B01L 3/502761; B01L 3/502784; G01N 1/28; G01N 33/487; B01L 2200/0636; B01L 2200/0652; B01L 2200/0673; B01L 2300/0816; B01L 2300/0851; B01L 2400/0475

(57) ABSTRACT

Microfluidic channels networks and systems are provided. One network includes a first fluid channel having a first depth dimension; at least a second channel intersecting the first channel at a first intersection; at least a third channel in fluid communication with the first intersection, at least one of the first intersection and the third channel having a depth dimension that is greater than the first depth dimension. Also provided is a flow control system for directing fluids in the network. Systems are additionally provided for flowing disrupted particles into a droplet formation junction, whereby a portion of the disrupted particles or the contents thereof are encapsulated into one or more droplets. Further provided is a method for controlling filling of a microfluidic network by controlling passive valving microfluidic channel network features.

### CROSS-REFERENCE

This Application is a Division of application Ser. No. 17/397,146 filed on Aug. 9, 2021. Application Ser. No. 17/397,146 is a Continuation of application Ser. No. 16/180,356 filed on Nov. 5, 2018, now U.S. Pat. No. 11,084,036. application Ser. No. 16/180,356 is a Continuation of Application PCT/US2017/032520 filed on May 12, 2017. Application PCT/US2017/032520 claims the benefit of U.S. Provisional Application 62/335,870 filed on May 13, 2016. The entire contents of these applications are incorporated herein by reference in their entirety.

### BACKGROUND

The field of microfluidics has advanced to the point that it is fulfilling much of its promise to supplant conventional laboratory fluid handling. The ability to precisely control the movement, accession, allocation, and mixing of minute amounts of fluids and subject those fluids to additional processing, analysis, and the like has helped move the field into the mainstream of scientific research, diagnostics, and medical devices.

As research and diagnostic needs become more and more complex, however, there is a need for the field of microfluidics to similarly advance in complexity, requiring a wide range of new functionalities within the microfluidic context. By way of example, microfluidic systems have been used to deliver and combine reagents within microfluidic channels and then perform subsequent processing and/or analytical operations on those reagents, including, e.g., thermal cycling, separations, optical, chemical or electrical detection, and a host of other operations.

In other applications, microfluidic systems have been used to partition small aliquots of aqueous fluids within flowing streams of immiscible fluids, e.g., oils, in order to compartmentalize reactions within those partitions for separate processing, analysis, etc. Specific implementations of these systems have been used to compartmentalize individual nucleic acids in order to perform quantitative amplification and detection reactions (qPCR).

In another implementation, discrete droplets in an emulsion contain both template nucleic acids and beads bearing large numbers of oligonucleotide barcodes, where a given bead will have a constant barcode sequence. The barcode is then used to prime replication of fragments of the template molecules within the particular partition. The replicate fragments created within a given droplet will all share the same barcode sequence, allowing replicate fragments from single long template molecules to be attributed to that longer template. Sequencing of the replicate fragments then provides barcode linked-reads that can be later attributed back to an originating long fragment, provide long range sequence context for shorter sequence reads.

With increasing demands on microfluidic systems, there is a need to add to the microfluidic tools that can be applied to expand their utility. The present disclosure provides a number of such tools and the uses and applications thereof.

### SUMMARY

The present disclosure provides novel, improved microfluidic structures, systems and methods for carrying out a variety of different fluid manipulations in microscale channel networks for use in a variety of different applications and methods.

In general a microfluidic channel network is provided, including: a first fluid channel having a first depth dimension; at least a second channel intersecting the first channel at a first intersection; at least a third channel in fluid communication with the first intersection, at least one of the first intersection and the third channel having a depth dimension that is greater than the first depth dimension.

In an aspect, the disclosure provides a microfluidic device. The microfluidic comprises a first fluid channel having a first depth dimension; at least a second channel intersecting the first channel at a first intersection; at least a third channel in fluid communication with the first intersection, at least one of the first intersection and the third channel having a depth dimension that is greater than the first depth dimension. In some embodiments, the microfluidic device further comprises a fourth channel segment, fifth channel segment, sixth channel segment and seventh channel segment intersecting the fourth channel segment at a second intersection, the fifth, sixth and seventh channel segments being coplanar, and where a cross sectional dimension of the seventh channel segment perpendicular to the first plane is larger than the cross sectional dimension of the fourth channel segment perpendicular to the first plane.

In some embodiments, the microfluidic device further comprises one or more steps disposed within one or more of the fourth and seventh channel segments, where the one or more steps provide the cross sectional dimension of the seventh channel segment that is larger than the cross sectional dimension of the fourth channel segment. In some embodiments, the one or more step increases the cross sectional dimension perpendicular to the first plane by at least 1%.

An additional aspect of the disclosure provides a microfluidic system. The microfluidic system comprises a microfluidic channel network comprising first, second, third and fourth channel segments in fluid communication at a first intersection, the first, second, third and fourth channel segments being coplanar, and where a cross sectional dimension of the fourth channel segment perpendicular to the first plane is larger than a cross sectional dimension of the first channel segment perpendicular to the first plane; and a flow control system for directing a first fluid through the first channel segment into the first intersection and into the fourth channel segment, and directing one or more focusing fluids from the second and third channel segments into the first intersection and into the fourth channel segment.

In some embodiments, the microfluidic system comprises a fifth, sixth and seventh channel segment intersecting the fourth channel segment at a second intersection, the fifth, sixth and seventh channel segments being coplanar, and where a cross sectional dimension of the seventh channel segment perpendicular to the first plane is larger than the cross sectional dimension of the fourth channel segment perpendicular to the first plane; and where the flow control system directs the first fluid and focusing fluids and second focusing fluids from the fifth and sixth channel segments into the second intersection. In some embodiments, the first fluid and focusing fluids flow in laminar flow into the fourth channel segment. In some embodiments, the microfluidic system further comprises one or more steps disposed within one or more of the channel segments and providing the larger cross sectional dimensions of the channel segments.

In some embodiments, the one or more step increases the cross sectional dimension perpendicular to the first plane by at least about 1%, at least about 5%, at least about 10%, at least about 20%, at least about 30%, at least about 40%, at least about 50% and least about 100%.

In another aspect, this disclosure provides a microfluidic system. The microfluidic system comprises first, second and third channel segments in fluid communication at a first intersection, the first, second, and third channel segments being coplanar, and where a cross sectional dimension of the third channel segment perpendicular to the first plane is larger than a cross sectional dimension of the first channel segment perpendicular to the first plane; and a flow control system for directing a first fluid through the first channel segment into the first intersection and into the fourth channel segment, and directing a second fluid from the second channel segment into the first intersection and into the third channel segment.

In some embodiments, the microfluidic system further comprises a fifth, sixth and seventh channel segment intersecting the fourth channel segment at a second intersection, the fifth, sixth and seventh channel segments being coplanar, and where a cross sectional dimension of the seventh channel segment perpendicular to the first plane is larger than the cross sectional dimension of the fourth channel segment perpendicular to the first plane; and where the flow control system directs the first fluid and focusing fluids and second focusing fluids from the fifth and sixth channel segments into the second intersection. In some embodiments, the first fluid and focusing fluids flow in laminar flow into the fourth channel segment.

In another aspect, the present disclosure provides a microfluidic system. The microfluidic system comprises a first channel segment fluidly connecting a source of disruptable particles, with a first droplet forming junction, the first channel segment comprising a constricted region proximal to the droplet forming junction; and a flow control system for driving the disruptable particles through the constricted region, where the constricted region comprises a cross sectional dimension reduced sufficiently to induce disruption of the disruptable particles driven through the constricted region, and for flowing disrupted particles into the droplet formation junction, whereby at last a portion of the disrupted particles or the contents thereof are encapsulated into one or more droplets.

In some embodiments, the microfluidic system further comprises a second channel segment, a third channel segment and a fourth channel segment in fluid communication with the first channel segment, where the second channel segment, third channel segment and fourth channel segment facilitate formation of one or more droplets at the droplet forming junction.

In some embodiments, the microfluidic system further comprises a fifth, sixth and seventh channel segment intersecting the fourth channel segment at a second intersection, the fifth, sixth and seventh channel segments being coplanar, and where a cross sectional dimension of the seventh channel segment perpendicular to the first plane is larger than the cross sectional dimension of the fourth channel segment perpendicular to the first plane; and where the flow control system directs the first fluid and focusing fluids and second focusing fluids from the fifth and sixth channel segments into the second intersection. In some embodiments, the first fluid and focusing fluids flow in laminar flow into the fourth channel segment.

In some embodiments, the microfluidic system comprises the constricted region positioned at a distance of fewer than 100 microns, fewer than 50 microns, fewer than 20 microns, fewer than 10 microns or fewer than 5 microns away from the droplet formation junction.

In some embodiments, the present disclosure provides a method of co-partitioning particles using the microfluidic system comprising providing one or more first particle and disrupting the particle by passage through the constriction; providing one or more second particle; and co-partitioning the first and second particle. In some embodiments, the first particle is one or more cells and the second particle is one or more bead. In some embodiments, the first particle is a single cell and the second particle is a single bead. In some embodiments, the bead is a gel bead. In some embodiments, the microfluidic system may further comprise co-partitioning a barcode. In some cases, the barcode is an oligonucleotide. In some cases, the oligonucleotide is a plurality of oligonucleotides having the same sequence. In some embodiments, the method of co-partitioning particles may further comprise providing a lysing agent. In some cases, the method of co-partitioning particles is performed without addition of a lysing agent.

In another aspect, the present disclosure provides a method for controlling filing of a microfluidic network. The method for controlling the filling of a microfluidic network comprises providing a microfluidic channel network comprising a first channel segment and a second channel segment intersecting the first channel segment at a first junction; providing a first fluid in the second channel segment up to the first junction, where capillary flow of the first fluid is interrupted at the first junction; providing a second fluid in the first channel segment, where the second fluid is capable of controlling filling of the microfluidic channel network by releasing the interrupted flow of the first fluid into the microfluidic channel network; and releasing the interrupted flow of the first fluid into the microfluidic channel network.

Some embodiments may provide the method for controlling the filling of a microfluidic network where the first channel segment comprises curved pinning points where the first channel segment meets the first junction. In some cases, the curved pinning points are configured and arranged to provide the interruption of capillary flow of the first fluid. In some embodiments, the method for controlling the filling of a microfluidic network is provided where the microfluidic channel network further comprises a third channel segment at the first junction, where the first fluid and second fluids flow in laminar flow into the third channel segment.

In some embodiments, the second fluid comprises a surfactant. In some embodiments, the surfactant concentration supports release of the interrupted capillary flow of the first fluid upon mixing of the first fluid and the second fluid.

In some embodiments, the microfluidic channel network further comprises one or more additional channel segments intersecting the third channel segment at a second junction, and where the released capillary flow of the first fluid is interrupted at the second junction. In some embodiments, the interruption of capillary flow of the first and second fluids at the second junction is the result of lower surfactant concentration in the mixed first fluid and second fluid. In some embodiments, the method for controlling the filling of a microfluidic network comprises the microfluidic channel network which further comprises a channel expansion feature arranged and configured to control the rate of flow of the first fluid into the microfluidic channel network. In some embodiments, the rate of flow of the first fluid is controlled to be reduced.

In another aspect, the present disclosure provides a method for controlling filing of a microfluidic network. The method for controlling the filling of a microfluidic network comprises providing a microfluidic channel network comprising a first channel segment and a second channel segment intersecting the first channel segment at a first junction; providing a first fluid in the first channel segment up to the first junction, where capillary flow of the first fluid is interrupted at the first junction; providing a second fluid in the second channel segment up to the first junction, where capillary flow of the second fluid is interrupted at the first junction; and providing pressure to both the first and second channel segments to control filling of the microfluidic channel network by releasing the interrupted flow of the first and second fluids into the microfluidic channel network.

In some embodiments, the method for controlling the filling of a microfluidic is where the first channel segment comprises a first curved pinning point and the second channel segment comprises a second curved pinning point where the first and second channel segments meet the first junction. In some cases, the curved pinning points are configured and arranged to provide the interruption of capillary flow of the first and second fluids. In some cases, the first and second curved pinning points each comprise a step feature. In some cases, the step features are configured and arranged to provide a smaller depth at the first junction compared to the depth of the first and second channel segments.

In some embodiments, the microfluidic channel network further comprises a third channel segment at the first junction. In some embodiments, microfluidic channel network further comprises one or more additional channel segments intersecting the third channel segment at a second junction, and where the released flow of the first and second fluids is interrupted at the second junction. In some cases, the method for controlling the filling of a microfluidic network comprises a microfluidic system network further comprising a channel expansion feature arranged and configured to control the rate of flow of the first fluid. In some cases, the rate of flow of the first fluid is reduced.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is an overview schematic illustration of a droplet formation microfluidic channel structure;

FIG. 2A is an overview schematic illustration of a microfluidic channel structure including steps and multiple junctions;

FIG. 2B is a side view schematic illustration of the structure in FIG. 2A;

FIG. 2C is a side view schematic illustration of an alternative configuration of the structure in FIG. 2A;

FIG. 3A is an overview schematic illustration showing flow of fluids in the structure of FIG. 2A;

FIG. 3B is a side view schematic illustration showing fluid flow in the structure of FIG. 3A;

FIG. 4A is an overview schematic illustration of a co-partitioning microfluidic channel network;

FIG. 4B is a close up schematic illustration detailing a side view of a constriction feature of FIG. 4A;

FIGS. 5A-D are schematic illustrations of a passive valving microfluidic channel structure in sequential stages of operation;

FIGS. 6A-D are schematic illustration of a passive valving microfluidic channel structure in sequential stages of operation;

FIGS. 7A-E shows an example system for generating droplets comprising beads and cell beads; and

FIG. 8 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “barcode,” as used herein, generally refers to a label, or identifier, that conveys or is capable of conveying information about an analyte. A barcode can be part of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time.

The term “subject,” as used herein, generally refers to an animal, such as a mammal (e.g., human) or avian (e.g., bird), or other organism, such as a plant. The subject can be a vertebrate, a mammal, a mouse, a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, or an individual that is in need of therapy or suspected of needing therapy. A subject can be a patient.

The term “genome,” as used herein, generally refers to genomic information from a subject, which may be, for example, at least a portion or an entirety of a subject's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach including ligation, hybridization, or other approaches.

The term “sequencing,” as used herein, generally refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina, Pacific Biosciences, Oxford Nanopore, or Life Technologies (Ion Torrent). As an alternative, sequencing may be performed using nucleic acid amplification, polymerase chain reaction (PCR) (e.g., digital PCR, quantitative PCR, or real time PCR) or isothermal amplification. Such devices may provide a plurality of raw genetic data corresponding to the genetic information of a subject (e.g., human), as generated by the device from a sample provided by the subject. In some situations, systems and methods provided herein may be used with proteomic information.

The term “bead,” as used herein, generally refers to a particle. The bead may be a solid or semi-solid particle. The bead may be a gel bead. The bead may be formed of a polymeric material. The bead may be magnetic or non-magnetic.

The term “sample,” as used herein, generally refers to a biological sample of a subject. The biological sample may be a nucleic acid sample or protein sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a check swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, scrum, urine, saliva, mucosal excretions, sputum, stool and tears.

The term “biological particle,” as used herein, generally refers to a discrete biological system derived from a biological sample. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell types, mycoplasmas, normal tissue cells, tumor cells, or any other cell type, whether derived from single cell or multicellular organisms. The biological particle may be obtained from a tissue of a subject. Biological particles may be disruptable particles.

The biological particle may be a hardened cell. Such hardened cell may or may not include a cell wall or cell membrane. The biological particle may include one or more constituents of a call, but may not include other constituents of the cell. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within a biological particle. The macromolecular constituent may comprise a nucleic acid. The macromolecular constituent may comprise deoxyribonucleic acid (DNA). The macromolecular constituent may comprise ribonucleic acid (RNA). The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise an oligonucleotide or polypeptide sequence. The molecular tag may comprise a DNA aptamer. The molecular tag may be or comprise a primer. The molecular tag may be or comprise a protein. The molecular tag may comprise a polypeptide. The molecular tag may be a barcode.

The efficiency of many single cell applications can increase by improving cell throughput. For example, this can be achieved by sorting a plurality of droplets that may or may not contain cells and/or particles therein to collect only the droplets that contain the cells and/or particles therein. The isolated population of droplets that contain the cells and/or particles therein can then be subject to further applications, such as nucleic acid amplification and/or sequencing applications.

### Microfluidic Structures, Systems and Methods for Droplet Generation

In an aspect, the present disclosure provides a microfluidic channel network. The microfluidic channel network may be used for generating droplets. The droplets may include biological samples and reagents necessary for processing the biological samples. In some examples, the droplets include beads comprising barcodes and biological particles comprising the biological samples, such as, for example, DNA and/or RNA. The biological particles may be cells comprising or enclosed in a gel or polymer matrix.

The microfluidic channel network may include a first fluid channel having a first depth dimension, at least a second channel intersecting the first channel at a first intersection, and at least a third channel in fluid communication with the first intersection. At least one of the first intersection and the third channel may have a depth dimension that is greater than the first depth dimension.

The microfluidic channel network may further comprise fourth channel segments, fifth channel segments, sixth channel segments and seventh channel segments intersecting the fourth channel segment at a second intersection. The fourth, fifth, sixth and seventh channel segments may be coplanar. A cross sectional dimension of the seventh channel segment perpendicular to the first plane may be larger than the cross sectional dimension of the fourth channel segment perpendicular to the first plane.

The microfluidic channel network may further comprise one or more steps disposed within one or more of the channel segments. The one or more steps may provide larger cross sectional dimensions of the channels. The one or more steps may increase the cross sectional dimension perpendicular to the first plane by at least about 1%, at least about 5%, at least about 10%, at least about 20%, at least about 30%, at least about 40%, at least about 50%, or at least about 100%. Such increase may be a gradual increase or a steep (or step) increase.

In another aspect, microfluidic system comprises a microfluidic channel network comprising first, second, third and fourth channel segments in fluid communication at a first intersection. The first, second, third and fourth channel segments may be coplanar. A cross sectional dimension of the fourth channel segment perpendicular to the first plane may be larger than a cross sectional dimension of the first channel segment perpendicular to the first plane.

The system may further comprise a flow control system for directing a first fluid through the first channel segment into the first intersection and into the fourth channel segment, and directing one or more focusing fluids from the second and third channel segments into the first intersection and into the fourth channel segment. A focusing fluid may be another aqueous stream or may be non-aqueous (e.g., oil). The flow control system may be or include one or more pumps for providing a negative pressure (e.g., pressure drop) to subject the first fluid to flow. Alternatively, the flow control system may be or include one or more compressors for providing positive pressure to subject a fluid (e.g., the first fluid) to flow.

At least a subset or all of the first, second, third and fourth channel segments may be coplanar (i.e., oriented along the same plane). As an alternative, at least a subset or all of the first, second, third and fourth channel segments may not be coplanar.

The microfluidic system may further comprise a fifth, sixth and seventh channel segment intersecting the fourth channel segment at a second intersection. Such channel segments may be channels or portions of channels. The fifth, sixth and seventh channel segments may be coplanar. A cross sectional dimension of the seventh channel segment perpendicular to the first plane may be larger than the cross sectional dimension of the fourth channel segment perpendicular to the first plane.

The microfluidic system may further comprise a flow control system that directs the first fluid and one or more focusing fluids and one or more additional focusing fluids from the fifth and sixth channel segments into the second intersection. The one or more additional focusing fluids may be the same or different from the one or more focusing fluids.

The first fluid and focusing fluids may flow in laminar flow into the fourth channel segment. As an alternative, the first fluid and focusing fluid may flow in turbulent flow into the fourth channel segment.

In another aspect, the microfluidic system may comprise a microfluidic channel network comprising first, second and third channel segments in fluid communication at a first intersection. The first, second, and third channel segments may be coplanar. A cross sectional dimension of the third channel segment perpendicular to the first plane may be larger than a cross sectional dimension of the first channel segment perpendicular to the first plane.

The system may further comprise a flow control system for directing a first fluid through the first channel segment into the first intersection and into the fourth channel segment, and directing a second fluid from the second channel segment into the first intersection and into the third channel segment. The flow control system may be or include one or more pumps for providing a negative pressure (e.g., pressure drop) to subject the first fluid to flow. Alternatively, the flow control system may be or include one or more compressors for providing positive pressure to subject a fluid (e.g., the first fluid) to flow. The microfluidic system may further comprise a fifth, sixth and seventh channel segment intersecting the fourth channel segment at a second intersection. The fifth, sixth and seventh channel segments may be coplanar. A cross sectional dimension of the seventh channel segment perpendicular to the first plane may be larger than the cross sectional dimension of the fourth channel segment perpendicular to the first plane.

The microfluidic system may further comprise a flow control system which directs the first fluid and focusing fluids and second focusing fluids from the fifth and sixth channel segments into the second intersection. The first fluid and focusing fluids may flow in laminar flow into the fourth channel segment.

The microfluidic system may further comprise a fifth, sixth and seventh channel segment intersecting the fourth channel segment at a second intersection, the fifth, sixth and seventh channel segments being coplanar and wherein a cross sectional dimension of the seventh channel segment perpendicular to the first plane is larger than the cross sectional dimension of the fourth channel segment perpendicular to the first plane; and wherein the flow control system directs the first fluid and focusing fluids and second focusing fluids from the fifth and sixth channel segments into the second intersection.

The first fluid and focusing fluids may flow in laminar flow into the fourth channel segment. Alternatively, the first fluid and focusing fluids may flow in turbulent flow into the fourth channel segment.

The microfluidic channel network may further comprise one or more steps disposed within one or more of the channel segments. The one or more steps may provide larger cross sectional dimensions of the channel segments. The one or more steps may increase the cross sectional dimension perpendicular to the first plane by at least about 1%, at least about 5%, at least about 10%, at least about 20%, at least about 30%, at least about 40%, at least about 50%, or at least about 100%. Such increase may be a gradual increase or a steep (or step) increase.

Reference will now be made to the figures, wherein like numerals refer to like parts throughout. It will be appreciated that the figures and features therein are not necessarily drawn to scale.

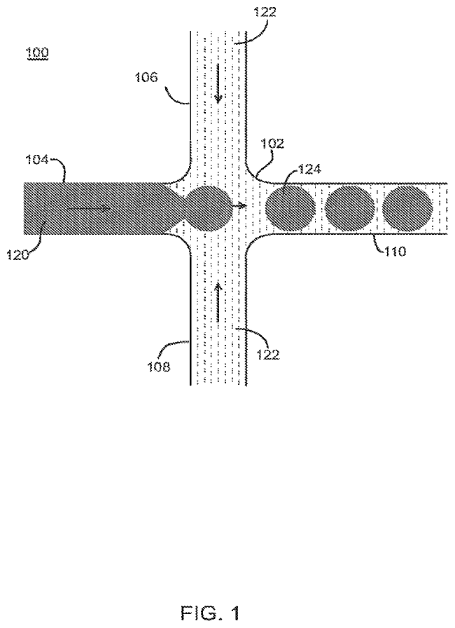

In a first example, provided are microfluidic channel networks and systems that provide enhanced partitioning of fluids, e.g., aqueous fluids partitioned as droplets in immiscible oils. For example, in some cases, channel networks may be provided that include a droplet generation junction at the intersection of a first aqueous fluid carrying channel segment and a first partitioning fluid, e.g., oil, carrying channel segment. Typically, aqueous droplets may be formed at the intersection as the aqueous fluid is dripped into the flowing oil stream at the intersection, forming droplets of aqueous fluid within the oil stream. FIG. 1 provides a simplified illustration of a droplet generation junction in which droplets of aqueous fluids are dripped into a non-aqueous fluid stream. As shown, a microfluidic channel network 100 includes a droplet generation junction 102 that may be coupled to an aqueous fluid channel segment 104. Two partitioning fluid inlet channels, 106 and 108, are also provided in fluid communication with the droplet generation junction 102. An outlet channel segment 110 may also be coupled to the droplet generation junction into which the droplets may be dripped into the non-aqueous partitioning fluid.

In operation (and as also illustrated in FIG. 1), an aqueous fluid 120 may be flowed into the droplet generation junction 102 from channel segment 104, while simultaneous streams of partitioning fluid 122 enter the junction from their respective channels 106 and 108. The focusing flow of the partitioning fluid constricts the aqueous fluid, which then drips off as droplets 124 into the flowing stream of partitioning fluid as it travels along the outlet channel segment 110. In some cases a point of constriction may be provided in the droplet generation junction in order to facilitate the dripping mechanism of droplet generation by accelerating the fluid flow through the junction.

Droplets of similar or substantially the same dimensions, e.g., cross section and/or volume, may be repeatedly formed. A number of factors can influence how such droplets are formed, including flow rates of the fluids that are interacting at the droplet generation junction, dimensions of the channels flowing into and out of the intersection, fluid characteristics of the fluids, and interactions between the fluids and the walls of the channels at or near the junction and in the downstream channels.

While certain immiscible phase emulsion arrangements like water in oil are discussed in respect to droplet formation herein, other emulsion arrangements in relation to the systems and methods described herein are envisioned, including but not limited to arrangements of immiscible phases such as air-in-water, oil-in-water, oil-in-water-in-oil, or the likes.

### Improved Flow Channel Structures

In some cases, including as illustrated above, droplets being formed at a droplet generation junction have been focused away from the side walls of the channels in which they are flowing by simultaneously flowing the non-aqueous partitioning fluid, e.g., oil, from opposing side channels, e.g., channel segments 106 and 108 in FIG. 1. While this may maintain a barrier stream of fluid between droplets and the side walls of the channel, because these microfluidic systems are fabricated in two dimensional planes, focusing flow tends to be only provided in those two dimensions. This leaves the possibility of droplets making contact with the upper and/or lower walls of a channel segment, with a possible result of a fluid contaminating the surface of the channels, and/or other adverse effects on droplet size and uniformity. Such contamination can be particularly problematic where partitions are used to contain different reagents or sample components, and cross-contamination may lead to aberrant results from downstream analyses. This potential problem is compounded where multiple reagents are combined and partitioned in these systems.

As described herein, in some cases, the droplet generation junction, e.g., FIG. 1 droplet generation junction 102, and/or the downstream channel segment coupled to the droplet generation junction, e.g., channel segment 110, are provided with a depth dimension that is greater than the depth dimension of the channel segments leading into the junction, e.g., channel segments 104, 106 and 108. It is envisioned that channels as shown in the figures presented need not be limited to uniform dimensions. Different height, width and length dimensions of the various channels described herein are envisioned. The dimensions may be uniform or different between channels or in respect to different portions of the same channel.

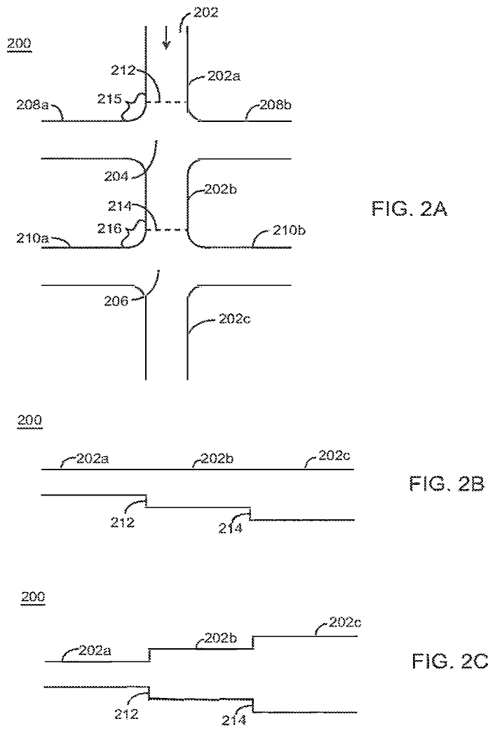

FIGS. 2A and 2B schematically illustrate an example of a microfluidic channel system that employs such channel structures. As shown in FIG. 2A (top view), a channel network 200 may include a main flow direction channel 202 made up of three distinct channel segments 202a, 202b and 202c, that may be divided by intersections 204 and 206. Intersection 204 may join channel segments 202a and 202b with side channel segments 208a and 208b. Likewise, intersection 206 may join channel segments 202b and 202c with side channel segments 210a and 210b. FIG. 2B is a profile view of the system.

FIG. 2C schematically illustrates a profile view of an additional example of a microfluidic channel system employing channel structures as described for FIGS. 2A and 2B.

As shown in FIGS. 2A-C, a first step structure 212 may be provided at the point at which channel segment 202a connects with intersection 204. Likewise a second step structure 214 may be provided at the point that channel segment 202b connects with intersection 206. The step structure may result in an increase in depth in moving from each of channel segments 202a and 202b into intersections 204 and 206, respectively. This is schematically illustrated in FIGS. 2B and 2C, which provide profile views of the channel network illustrating the change in depth dimension as channel segment 202a expands into segment 202b and 202c at intersections 204 and 206, respectively.

The step structures described herein may exist at one or both of the upper and/or lower channel walls, and may result in an increase in the depth dimension of the channel, e.g., the dimension perpendicular to the main plane of a device, of at least about 1%, at least about 5%, at least about 10%, at least about 20%, at least about 30%, at least about 40%, at least about 50%, and in some cases by at least about 100% or more.

While steps can be provided as structural pinning features, it is envisioned that the same or similar effects can be achieved using a material-based “step”, for example, by patterning sections of channels using a variety of compounds, compositions or even texture. For example, in a channel that has been patterned chemically to form well-defined sections of hydrophilic and hydrophobic surface conditions, an aqueous fluid flowing across a hydrophilic section will pin at an interface with a hydrophobic section.

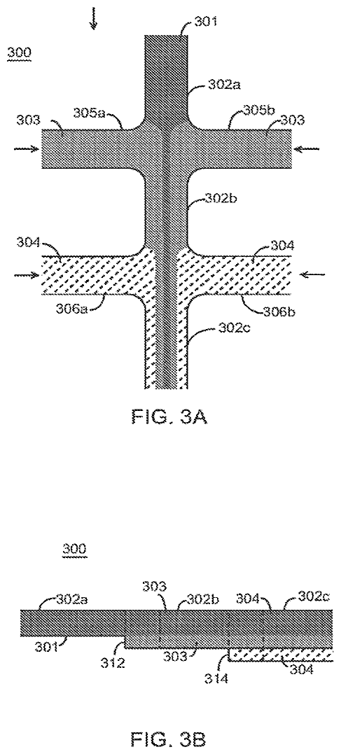

FIGS. 3A and 3B give an example of the FIGS. 2A and 2B channel networks in operation. Fluids introduced into a given intersection, e.g., from channel 302a into intersection 306, may remain separated from the lower channel walls by virtue of the presence of the fluid 303 brought into the intersection from channel segments 305a and 305b. FIG. 3A shows an example of an aqueous fluid in 303 being used as a focusing fluid to narrow the stream of the fluid in 301. Likewise, the combined fluids flowing into channel segment 302b may be displaced from the lower channel wall by fluids flowed into intersection 308 by the fluid 304 brought in from channel segments 306a and 306b. Although described in some cases in terms of the intersections being droplet generation junctions, such step structures may also be useful in maintaining laminar flow separation of combined fluids that are not at a droplet generation junction, but merely at a mixing or fluid combination intersection. For example, two or more different aqueous fluids may be brought together at intersection 308 via channel segments 302a-c, 305a-b and 306a-b. FIG. 3B details the flow of fluids 301, 303 and 304 in this fashion. By virtue of the presence of the step structures 312 and 314 indicated in FIG. 3B, any adverse interactions between one or more of the aqueous fluids and side walls, e.g., adsorption of sample components, may be reduced or avoided as the focusing flows may shield such fluids from the side, upper and/or lower channel walls.

As the combined fluid is flowed into intersection 308, immiscible partitioning fluid may be introduced from each of side channels 304a and 304b, to further surround the combined fluid stream. This is illustrated in a side view in FIG. 3B, where the partitioning fluid is illustrated as the white space around the cell fluid and lysis agent. As will be appreciated, at this intersection, the fluids may be partitioned into droplets of cell suspension combined with lysis agent, surrounded by the partitioning fluid. By incorporating the step, as well as the focusing function of the fluids from the side channels, one may minimize the level of contamination of the side walls from the first fluid introduced, e.g., the lysis agent/surfactant. As will be appreciated, the ability to avoid surface contamination with other surfactants, e.g., that one is using as a lysis agent as described in greater detail herein, is of significant importance in droplet based partitioning systems, which rely on specific surfactant compositions to ensure proper partitioning and subsequent stability of aqueous droplets in oil based fluids. Likewise it will be appreciated that other side wall contaminants such as polymers and lipids may be avoided.

### Co-Partitioning Channel Networks

In another aspect, microfluidic system is described. The microfluidic system may comprise a first channel segment fluidly connecting a source of disruptable particles, with a first droplet forming junction. The disruptable particles may be a single cell or multiple cells from a biological specimen.

The first channel segment may comprise a constricted region proximal to the droplet forming junction. A flow control system may further drive the disruptable particles through the constricted region. The constricted region may comprise a cross sectional dimension reduced sufficiently to induce disruption of the disruptable particles driven through the constricted region. The disruptable particles may then physically become disrupted, damaged or lysed upon passage through the channel, resulting in a damaged or lysed cell. The flow control system may then be used to flow the disrupted particles into the droplet formation junction, whereby a portion of the disrupted particles or the contents thereof may be encapsulated into one or more droplets.

The microfluidic system may further comprise a second channel segment, a third channel segment and a fourth channel segment in fluid communication with the first channel segment. The second channel segment, third channel segment and fourth channel segment may facilitate the formation of one or more droplets at the droplet forming junction.

The microfluidic system may further comprise fifth channel segments, sixth channel segments and seventh channel segments intersecting the fourth channel segment at a second intersection. The fifth, sixth and seventh channel segments may be coplanar. A cross sectional dimension of the seventh channel segment perpendicular to the first plane may be larger than the cross sectional dimension of the fourth channel segment perpendicular to the first plane. The flow control system may direct the first fluid and focusing fluids and second focusing fluids from the fifth and sixth channel segments into the second intersection.

The first fluid and focusing fluids may flow in laminar flow into the fourth channel segment. As an alternative, the first fluid and focusing fluid may flow in turbulent flow into the fourth channel segment.

The microfluidic system may position the constricted region at a distance fewer than 100 microns, 50 micrometers (microns), 20 microns, 10 microns, 5 microns 1 micron, 0.5 microns, or less away from the droplet formation junction.

In addition, a method of co-partitioning particles is provided using the microfluidic system described above. The method may comprise providing a first particle (e.g., cell from a biological specimen) and disrupting the particle by passaging through the constricted region. The microfluidic system may further provide a second particle (e.g., bead/beads which may contain barcodes and/or other reagents); and co-partitioning the first and second particle in to one/more droplets for further processing of the biological specimen. Alternatively, the first particle may comprise multiple cells. The second particle may also comprise multiple beads to be co-partitioned with the first particle comprising multiple cells.

The microfluidic system may enable co-partitioning the first and the second particle in the microfluidic system, where, the second particle may comprise a gel bead. Additionally, the second particle may comprise a barcode. The barcode may comprise an oligonucleotide. Alternatively, the barcode may comprise a plurality of oligonucleotides having the same sequence. In another embodiment, the first or the second particle may comprise a lysing agent. In yet another embodiment, the method of co-partitioning the first and the second particle may be performed without the addition of a lysing agent.

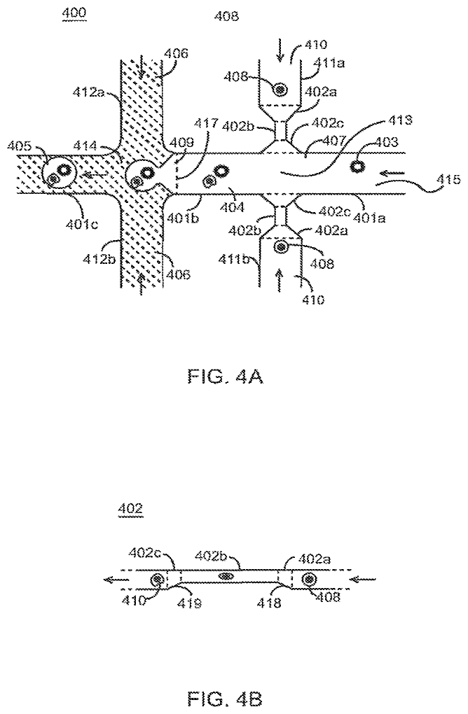

FIGS. 4A and 4B schematically illustrate an example of an operation in which disruptable particles, e.g., cells, are co-partitioned with other reagents and or particles in an iterative fluid combining step using the channel networks related to those described above, including laminar flow of fluids and use of step features. Exemplary reagents may include, e.g., lysis agents such as an effective concentration of a surfactant and exemplary particles may include, e.g., other cells or beads such as gel beads.

As shown in FIG. 4A, with reference to the microfluidic channel networks illustrated in FIGS. 2A and 3A, a first fluid 415 which can be aqueous, may include a bead 403, may be flowed along channel segment 401a and into intersection 413. Intersection 413 may also be in fluid communication with one or more other channel segment. As illustrated in FIG. 4A, the channel intersecting is a constriction 402 feature comprised of sections 402a-c. As illustrated there may be two channels intersecting channel 401a at intersection 413, but it should be understood that a single or three or more channels may intersect.

The constriction 402 feature is detailed in FIG. 4B. As shown, it may be comprised of a channel 402a having a first depth leading to a ramp or step 418 feature that connects to a channel 402b having a smaller second depth and/or width. Channel 402b may be connected to a second ramp or step 419 feature that connects to a channel 402c having a depth similar or the same as for channel 402a. As shown, disruptable particles, e.g., a cell or cells 408 may be introduced into the constriction 402 feature and physically become disrupted, damaged or lysed upon passage through channel 402b, resulting in a damaged or lysed cell 410.

As shown in FIG. 4A, disruptable particles, e.g., cell or cells 410 may be introduced to constriction feature 402 through channel 411a and 411b in a second fluid 410, which may be aqueous. As fluid 410 and cells 408 encounter the constriction 402, the cell or cells may be damaged or lysed, then mix with aqueous fluid 415 at intersection 413, where in some cases a bead 415 can be associated with the damaged or lysed cell 410. A cell, e.g., a single cell, may be associated with a bead to form a lysed cell-bead complex 404, either at intersection 413 or as passing along channel 401b. Channel 401b connects to one or more additional channels at intersection 414. As shown in FIG. 4A, three channels, 412a, 412b and 401c may connect with channel 401b at intersection 414. As indicated by hash marks, channels 412a and 412b may bring a third partitioning fluid 406 (hash marked) to intersection 414 for interaction with the fluids 415 and 410 surrounding the lysed cell-bead complex 404. As indicated at 409, droplet or partitioning fluid 406 may be immiscible with fluids 415 and 410, creating a partitioned lysed cell-bead complex 405. As shown, the partitioned lysed cell-bead complex 405 may flow along channel 401c for subsequent purposes.

### Cell Content Analysis

As noted above, in some cases, microfluidic channel networks are particularly desired for use in analyzing the contents of cells, and particularly for evaluation of the contents of individual cells. In certain cases, an individual cell may be partitioned within a single droplet of aqueous fluid in an immiscible partitioning fluid. By co-partitioning a lysis agent, e.g., as described above, along with the cell, one may disrupt the cell and release its contents into the droplet for subsequent processing and/or analysis within the droplet. For example, as described in co-pending U.S. Patent Application Publication No. 2015/0376609, filed on Jun. 26, 2015, which is entirely incorporated herein by reference, an individual cell may be co-partitioned with a lysis agent and a set of oligonucleotide barcodes, as described above. The lysis agent may then act on the cells to release the contents of the cell into the partition. The co-partitioned barcodes may then be used to tag the nucleic acid contents of the cell as described above. Different barcode sequences may be added to different droplets or partitions within the overall emulsion, such that nucleic acids from a given cell will only be tagged with one barcode, allowing more effective attribution of the barcodes, and their connected nucleic acids, to an originating cell, once those nucleic acids and barcodes are sequenced.

In some cases, as shown in and discussed above for FIGS. 4A and 4B above, it may be desirable to forego the use of a lysis agent, or provide additional efficacy to the lysis step of the overall process through the inclusion of a mechanical lysis structure within the channels of the microfluidic device. However, the same mechanical forces that may result in disruption of a cellular membrane, may also result in disruption of fluid forces (e.g., surface tension) holding a partitioned droplet together. As such, as described herein, the lysis structure may be included proximal to, but upstream from the droplet forming junction at which it is desired to partition the cell contents.

It is worth noting that while the systems and methods disclosed herein are capable of performing cell lysis, even complete cell lysis, it is envisioned and can be understood that modifications to the lysis structures or lysis agent used can result in any desired degree of partial lysis or disruption of the cell, e.g., disruption of a cell membrane. For convenience however, the phrases lysis structure and lysis agent will be used to describe any level of cell disruption.

A particular advantage of the systems and methods disclosed herein includes the ability to lyse a cell, e.g., a single cell, immediately upstream of the droplet generation junction, so one can be more certain that the contents of a given cell will be partitioned within a single droplet (or a small number of droplets). Moreover, because of the laminar flow characteristics of microfluidic systems, one may be reasonably certain that only minimal diffusion of the cellular contents will occur. It is envisioned that minimal diffusion or dispersion of cell contents may be achieved by providing short residence times (the time between lysis and encapsulation for example). In some cases, the lysis structure may be provided within a distance of fewer than 100 microns, fewer than 50 microns, fewer than 20 microns, fewer than 10 microns, fewer than 5 microns away from the droplet generation junction. In other cases, the lysis structure may be provided at a distance that provides for minimal amount of diffusion time for the released contents of a cell between lysis and partitioning within the droplet generation junction at the flow rates used in a given operation. For example, in some cases, this time may be less than about 100 milliseconds (ms), less than about 50 ms, less than about 30 ms, less than about 20 ms, less than about 10 ms, or even less than about 5 ms down to as low as 1 ms, 0.5 ms, 0.1 ms or even less than about 0.01 ms.

In some cases, a lysis structure may include a cross-sectional restricted region of a channel that imparts sufficient shear forces upon a cell or other particle so as to cause its disruption under the conditions being applied, e.g., flow rates, pressures, presence of other lysis reagents, etc. An example of such a structure is illustrated in FIGS. 4A and 4B. As shown, a constricted channel segment 402 through which cells are passed prior to entering into a droplet generation junction, may be provided with a significantly reduced cross section, in at least one dimension, and in some cases, two dimensions. In such cases, transition of flowing streams through such constricted spaces may subject cells within those streams to high shear stresses than can result in lysis of those cells. As noted, this may be accompanied by the presence of other lysis forces, such as lysis agents present in the cell suspension.

In some cases the lysis structure may include a series of constrictions, e.g., at least 2, at least 3, at least 4, at least 5, etc. or more constrictions provided in a series. In other cases the lysis structure may include pillar features arranged in a pathway a cell or cells may traverse and in so doing suffer partial or complete lysis. Combinations of the above lysis structures are also envisioned.

### Passive Valving Structures—Aqueous Channels at Intermediate Positions

The present disclosure provides methods for controlling filling of a microfluidic network. A method for controlling filling of a microfluidic channel network may comprise providing a first channel segment and a second channel segment intersecting the first channel segment at a first junction. The first channel segment and second channel segment may be part of a device, such as an integrated device. The device may be a microfluidic device. The device may be a droplet generator. The device may be part of a system. Such system may include a controller for regulating, for example, fluid flow. The system may include one or more pumps and/or one or more compressors (or other actuators) for facilitating fluid flow.

A first fluid may be provided in the second channel segment up to the first junction. In this embodiment the capillary flow of the first fluid may be interrupted at the first junction. A second fluid may also be provided in the first channel segment. The second fluid may be capable of controlling filling of the microfluidic channel network by releasing the interrupted flow of the first fluid into the microfluidic channel network. This may assist in releasing the interrupted flow of the first fluid into the microfluidic channel network.

In some cases, the first channel segment comprises curved or angled pinning points where the first channel segment meets the first junction. Such curved pinning points may be configured and arranged to provide the interruption of capillary flow of the first fluid.

The device may further comprise a third channel segment at the first junction. The first fluid and second fluids may further flow in laminar flow into the third channel segment. Alternatively, the first fluid and second fluids may further flow in turbulent flow in to the third channel.

In some cases, a second fluid comprising a surfactant may be used. The surfactant concentration in the second fluid may be adjusted to support the release of the interrupted capillary flow of the first fluid upon mixing of the first fluid and the second fluid. The interruption of capillary flow of the first and second fluids at the second junction may be the result of a lower surfactant concentration in the mixed first fluid and second fluid. The mixed first and second fluids may be interrupted by either decreased surfactant concentration due to mixing or due to physical pinning of meniscus which may be provided at a step change in channel depth and or channel width.

The microfluidic channel network may further comprise one or more additional channel segments. Such additional channel segments may intersect the third channel segment at a second junction. The released capillary flow of the first fluid may further be interrupted at the second junction.

The device may comprise a microfluidic channel network further comprising a channel expansion feature. This channel expansion feature may be arranged and configured to control the rate of flow of the first fluid into the microfluidic channel network. The channel expansion feature may control the rate of flow of the first fluid. The channel expansion feature may reduce the flow rate of the first fluid. Alternatively, in yet another embodiment, the microfluidic channel expansion may increase the flow rate of the first fluid.

In yet another aspect, a method for controlling filing of a microfluidic network is described. The microfluidic network may comprise providing a microfluidic channel network comprising a first channel segment and a second channel segment intersecting the first channel segment at a first junction. The microfluidic channel network may provide a first fluid in the first channel segment up to the first junction. Capillary flow of the first fluid may be interrupted at the first junction. Additionally, it may provide a second fluid in the second channel segment up to the first junction. Capillary flow of the second fluid may also be interrupted at the first junction. The method may then further include providing pressure to both the first and second channel segments to control the filling of the microfluidic channel network by releasing the interrupted flow of the first and second fluids into the microfluidic channel network.

The microfluidic network may further comprise a first channel segment comprising a first curved pinning point and the second channel segment comprising a second curved pinning point where the first and second channel segments meet the first junction. The curved pinning points in both the first channel segment and the second channel segment may be configured and arranged to provide the interruption of capillary flow of the first and second fluids.

In some cases, the first and second curved pinning points in the first channel segment and the second channel segment respectively may each comprise a step feature. Additionally, the step features may be configured and arranged to provide a smaller depth at the first junction compared to the depth of the first and second channel segments. In some cases, the microfluidic channel network may further comprise a third channel segment at the first junction. These additional channel segments may intersect the third channel segment at a second junction, wherein the released flow of the first and second fluids is interrupted at the second junction.

Some embodiments of this method of controlling the filling of the microfluidic network may further comprise a channel expansion feature. This channel expansion feature may be arranged and configured to control the rate of flow of the first fluid. The channel expansion feature may control the rate of flow of the first fluid. The channel expansion feature may reduce the flow rate of the first fluid. Alternatively, in some cases, the microfluidic channel expansion may increase the flow rate of the first fluid.

As described elsewhere herein, microfluidic channel networks may be used in conjunction with different types of fluids within the same channel network, including different types of aqueous fluids, aqueous and non-aqueous fluids, etc. In such systems, as well as in many other applications of microfluidic systems, it may be desirable to provide stepwise additions of different fluid materials to the channel networks, in order to rely on capillary action and wicking to fill the channels, while only having such filling reach certain portions of the channel network and not others.

In certain cases, microfluidic structures or arrangements may be provided to ensure proper and selective filling of different channel segments. One example of such an arrangement incorporates an intervening passive valving structure disposed between two channel segments in which differential filling is desired. In a simple context, such valving structures may include areas of increased cross sectional dimension disposed at an end of a given channel segment, such that capillary forces can draw fluid into the valving structure. These structures may include step structures that increase the depth of the channel, widened channel regions that increase the lateral cross section of the channels or combinations of these. In some cases, the passive valving may be provided at intersections with connected channel segments, which provide the increased cross sectional area.

Despite the use of these passive valving structures, in many cases, additional measures may be used to prevent fluid wicking beyond a desired point. For example, in certain cases, fluids may be disposed within channel segments that include concentrations of surfactants, e.g., cither for partitioning or for use as cell lysis agents. Such surfactant laden fluids can, in some instances, be prone to wicking through microfluidic channel networks despite the presence of passive valves, e.g., by decreasing the contact angle between the fluid and the surface of the channel, resulting in a higher capillary pressure. As such, in using such passive valving structures it may sometimes be desirable to incorporate additional measures to avoid such unintended wicking. In one exemplary approach, an intervening aqueous fluid that may be low in surfactant, e.g., has a higher contact angle than the surfactant laden fluid, may be provided at a passive valving structure. When the surfactant laden fluid reaches the passive valve and adjacent aqueous fluid barrier, the surfactant may be diluted and the contact angle at the interface may be increased such that the passive valve structure functions as desired, preventing further wicking into the channels of the device.

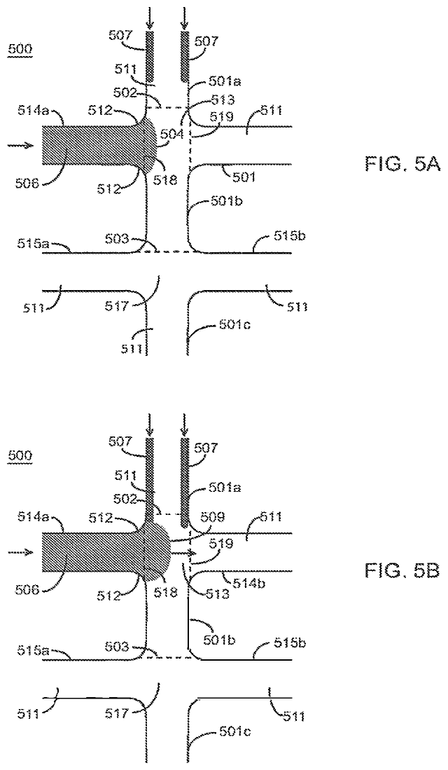

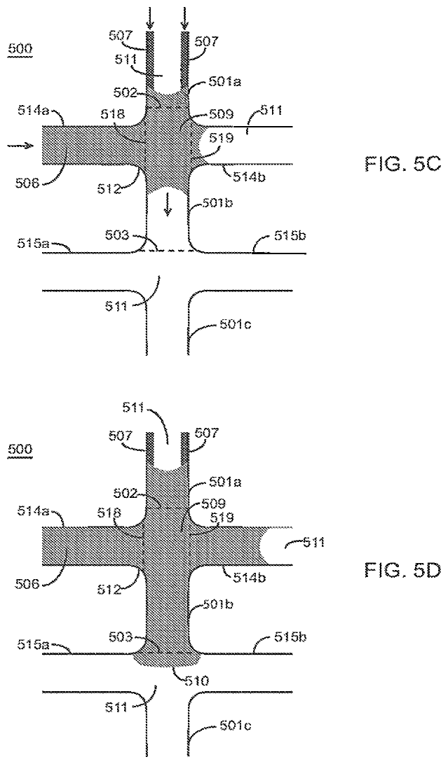

FIG. 5 is a schematic illustration of an approach to such valving structures, useful, for example, in the context of a droplet generating microfluidic device in which aqueous fluids are partitioned into non-aqueous fluids as an emulsion. As noted, in many cases, these systems may include concentrations of surfactants mixed into one or both of the aqueous and non-aqueous fluids within the channel network in order to facilitate partitioning of fluids within the channel network, e.g., at a droplet generation junction or the like.

In some cases, additional elements may be incorporated into these intersecting channel segments to allow for segmented introduction of the differing fluids into the device. In some cases, two channel segments in which different filling is desired may be joined at an intersection with a third channel segment. The third channel segment may be first filled with a fluid that will provide a break in the capillary action on one or more fluids introduced into the two other channel segments. FIG. 5 provides one example of such a valving arrangement. As will be appreciated, one, two, three, four or more such structures may be provided within a microfluidic device in order to facilitate differential fluid introduction. Furthermore, the additional structures may be arranged at a variety of angles and provided in staggered or other configurations.

In providing a droplet generation junction within a microfluidic channel network, e.g., as described above, it is generally desirable to provide the non-aqueous fluids in the downstream channels without them wicking into the channels that are to deliver aqueous fluids into the droplet generation junction. Due to the high wettability of non-aqueous fluids on material surfaces, the only way to prevent contamination of the upstream, aqueous fluid-delivering channels with non-aqueous fluid is to fill the desired channels with the aqueous fluids before introducing the non-aqueous fluid. Hydrostatic pressure may then be used to passively balance or counteract the capillary pressure associated with an oil-aqueous or oil-air meniscus.

FIGS. 5A-D illustrate an exemplary passive valving structure arrangement and the process of operating a passive valve. FIG. 5A shows an initial resting state of the passive valving in structure 500. Fluid 506, which can be a surfactant poor aqueous fluid, is shown within channel 514a and may be pinned at intersection 513, which is filled with air. Pinning point 512 serves to hold the fluid in a primed fluid state shown as menisci 504 against air 511. A second fluid 507, which may be surfactant-rich, is shown entering channel 501a and approaching the primed valve fluid 504. It should be understood that surfactant-rich fluid traveling quickly through the corners/gutters of the channels may be useful. In particular, the second fluid 507 “tendrils” of the surfactant-rich fluid move faster than the bulk surfactant-rich fluid. This feature may control the dilution of surfactant-rich with surfactant-free, and thus the stability of the pinning of the combined fluid at pinning point 513 (higher stability may be achieved at higher dilution). The shape of the cross-section is a useful design feature for controlling the speed of the second fluid 507 “tendrils”, which can be faster than the source fluid within the channel. For example, the shape may be polygonal, trapezoidal, rectangular, square, etc. (not shown).

The pinning point 512 may be configured and arranged, e.g., as curved corners at intersection 513. The radii of curvature may be adjusted to optimize the action of pinning point 512 as desired, the action being in a range from slight to strong restraint of fluid 506 at pinning points 512. Physical or chemical steps may be included within channels or at intersections of channels as required to provide and enable pinning points as described herein. For example, as illustrated in FIGS. 5A-D, steps 502, 503, 518 and/or 519 can be included.

FIG. 5B shows the releasing of the primed passive valve as fluid 509 extends further into intersection 513. The release of fluid at the passive valve was triggered by fluid 507 when it reached and contacted fluid 504 as shown in FIG. 5A. In some cases, after crossing step 502 in channel 501a, fluid 507 may touch and fluidically communicate with fluid 504 of FIG. 5A, resulting in pin release and the protrusion of fluid face 509.

FIG. 5C shows the flow of fluid 506 extending into intersection 513 as well as into channels 501a, 514b and 501b and displacing air 511. FIG. 5D shows further flow of fluid 506 beyond step 503 in channel 501b, to pinning point 516 to form fluid face 510 at intersection 517 against air 511. Channels 515a, 515b and 501c may remain air 511 filled, flow of fluid 507 is stopped and structure 500 may be pinned at intersection 517.

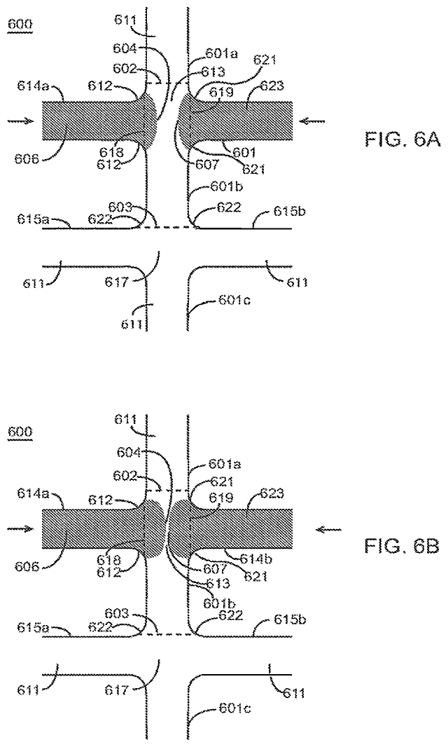

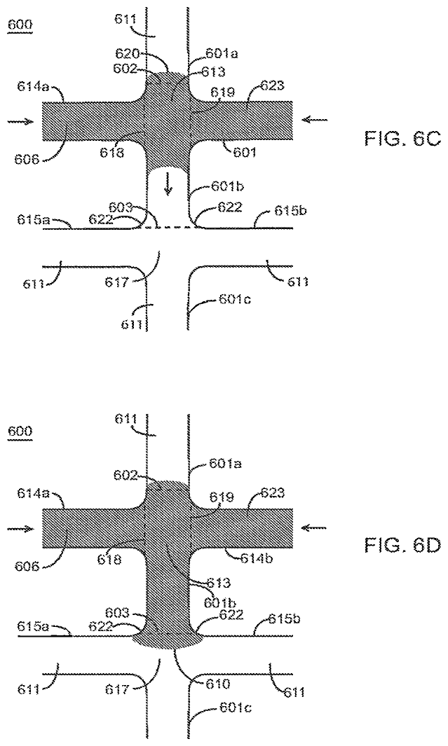

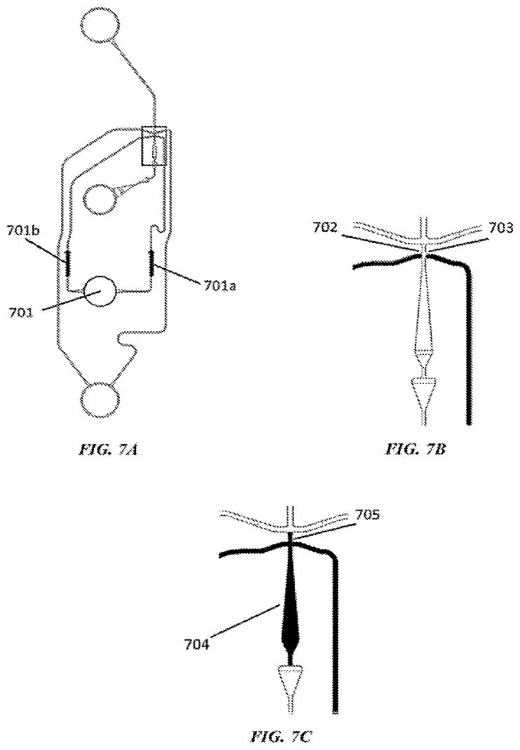

FIG. 6 is a schematic illustration of an alternative approach to providing pinning, useful, for example, in the context of a droplet generating microfluidic device in which aqueous fluids are partitioned into non-aqueous fluids as an emulsion.

FIGS. 6A-D illustrate an example of a pinning structure arrangement and the process of its operation. FIG. 6A shows an initial pinned state of the fluids in structure 600. First fluid 606 and second fluid 623, which may be surfactant-poor aqueous fluids, are shown within channels 614a and 601 respectively, and are pinned at intersection 613, that may be filled with air 611. Pinning points 612 and 621 serve to hold the fluid in a primed fluid state shown as menisci 604 against air at intersection 613. The pinning points 612 and 621 may be configured and arranged, e.g., as curved corners at intersection 613. The radii of curvature may be adjusted to optimize the action of pinning points 612 and 621 as desired, the action being in a range from slight to strong restraint of fluids 606 and 623 at pinning points 612 and 621 respectively.

Physical and chemical steps may be included within channels or at intersections of channels as required to provide and enable pinning points as described herein. For example, as illustrated in FIGS. 6A-D, steps 602, 603, 618 and/or 619 may be included. Physical steps may provide downward or upward or ramped sections within the systems depending on the optimal action of pinning, the action being in a range from slight to strong. For example, steps 618 and 619 may be raised steps that provide a shallower depth at intersection 613 than for channels 6 14a and 601, to provide a slight pinning action. Chemical steps may provide regions of high or low hydrophobicity by treatment with different compounds, e.g., fluorinated compounds for high hydrophobicity, depending on the optimal action of pinning, the action being in the range from slight to strong.

FIG. 6B shows the releasing of the primed pinning points as fluids 606 and 623 extend further into intersection 613. The release of fluid at the pinning points may be triggered, for example, by hydrostatic pressure acting on the fluid or by design of the pinning points to provide only a slight restraint of fluids 606 and 623. After a desired time (e.g., 3, 5, 10, 30, 60, 120, 240 seconds), the menisci may merge and the merged liquid may be re-pinned at a desired location.

The time required for the menisci to merge may be controlled by the hydrostatic pressure acting on the fluids, by the resistance of the microchannels through which the liquids flow, and/or by the aspect ratio of the channels near the junction. For example, when the channels in which the fluids reside are wider and/or deeper than the channel across which they merge, once pinned at the intersection, the menisci may have a shorter distance to travel before they meet. Wider and/or deeper channels may accommodate greater curvature of the pinned menisci, which may allow them to extend further into the intersection. If the fluids wet the channel surface, decreasing the channel depth in the region shown (the region can also extend further down the channels, up to a point where it starts to increase the resistance of the channels appreciably, which may be undesired when it increases the merging time too much) may generate a stronger capillary pressure in the region, which may accelerate the speeds at which the menisci travel and may cause them to merge faster.

After merging of menisci 604 and 621, the combined fluids 606 and 623 may flow further into the structure 600. FIG. 6C shows the flow of the combined fluids extending along channel 601a and 601b, beyond intersection 613. Step 602 may include pinning of combined fluids. Step 602 may include meniscus 620.

FIG. 6D shows further flow of the combined fluids beyond step 603 in channel 601b, to pinning point 622 to form meniscus 610 at intersection 617 against air 611. Channels 615a, 615b and 601c may remain air 611 filled.

In a further embodiment, stagnation zones that may occur at, for example, pinning points 612 and 621 shown in FIGS. 6A and 6B as a result of steps and may potentially trap undesirable air bubbles. To prevent such bubble formation a pulse of pressure may be applied to the microfluidic channel network to displace air into a well, reservoir, channel or other component of the network.

Additional features may be included in any of the microfluidic channel networks described herein. Features may include, but are not limited to, constrictions, expansions, steps, coatings, etc. One particularly useful feature is an expansion feature for controlling flow rate. In one embodiment the flow rate may be slowed by inclusion of one or more expansion feature in a given channel or at a junction of channels. The expansion feature may be configured in any of a number of ways but generally is provided as a widening or expansion of a portion of a channel or intersection. The shape of the expansion feature may be regular, irregular, short, elongated, staggered, etc. In one embodiment the expansion feature may be trapezoidal in shape. In another embodiment the expansion feature may be triangular, for example, scalene, isosceles, acute, right, equilateral or obtuse.

Channel networks of the present disclosure may include multiple steps, such as at least 1, 2, 3, 4, 5, 6, 7, 8, 9, 10, 20, 30, 40, 50, 60, 70, 80, 90, or 100 steps. The steps may be oriented in order of increase cross-section.

A channel network of the present disclosure may be disposed in an integrated device, such as a microfluidic device (e.g., a cartridge or chip). Such device may be consumable (e.g., a single-use device, which may be disposed). Alternatively, a channel network of the present disclosure may be disposed in multiple devices. Such devices may be configured to integrate with a system that is configured to, for example, facilitate fluid flow.

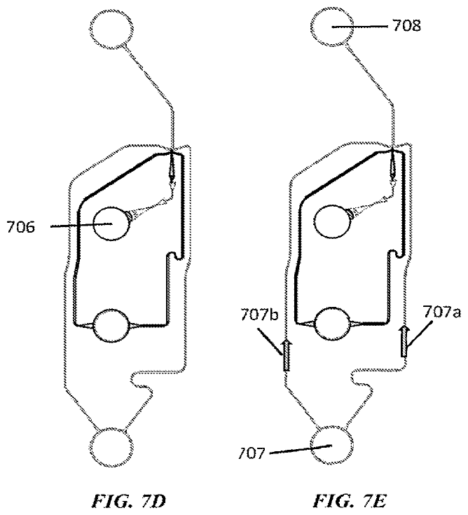

FIGS. 7A-E schematically illustrates an example operation using both passive valving and pinning structure arrangements. FIG. 7A shows an initial resting state of fluid 701. Fluid 701, which may be a surfactant poor (e.g., less than 10%, 5%, 4%, 3%, 2%, 1%, or less (e.g., not detectable) concentration of a surfactant) aqueous fluid containing biological specimens or biological particles (e.g., cells) is shown within channels 701a and 701b. The fluid 701 may, for example, biological particles, such as, for example, one or more cells. Such biological particles may include one or more cells. Each of the one or more cells may include or be enclosed within a gel or polymer matrix.

FIG. 7B shows pinning points which may serve to hold the fluid in a primed fluid state shown as menisci 702 and 703. The pinning points may be configured and arranged, e.g., as curved corners at the intersection. The radii of curvature may be adjusted to optimize the action of pinning points as desired, the action being in a range from slight to strong restraint of fluid 701 at the pinning points.

In some examples, the radii of curvature may be from about 1×10⁸ to 1×10⁻¹ meters (m), from about 1×10⁻⁷ to 1×10⁻² m, from about 1×10⁶ to 1×10⁻³ m, from about 1×10⁻⁶ to 1×10⁻⁴ m, or from about 1×10⁻⁶ to 1×10⁻⁵ m.

FIG. 7C shows the merging of menisci 702 and 703 and the combined fluids flow further in to the passive valve structure 704. Fluid 701 may also be pinned at the generation step junction 705.

FIG. 7D shows the stepwise addition of a surfactant laden fluid 706. This fluid may comprise beads that comprise further of barcodes and/or other reagents needed for further processing of the biological sample 701. Air or other fluid (e.g., gas) trapped between 701 and 706 may prevent contacts of fluids and may prevent the surfactant laden fluid from wetting the generation region.

FIG. 7E shows the stepwise addition of the fluid 707 which may be used as a partitioning fluid. Fluid 707 may flow through the channels 707a and 707b to fluidically interact with the fluids 701 and 706 at the junction 705. These combinations of fluids may further flow in to channel 708.

### Computer Control Systems

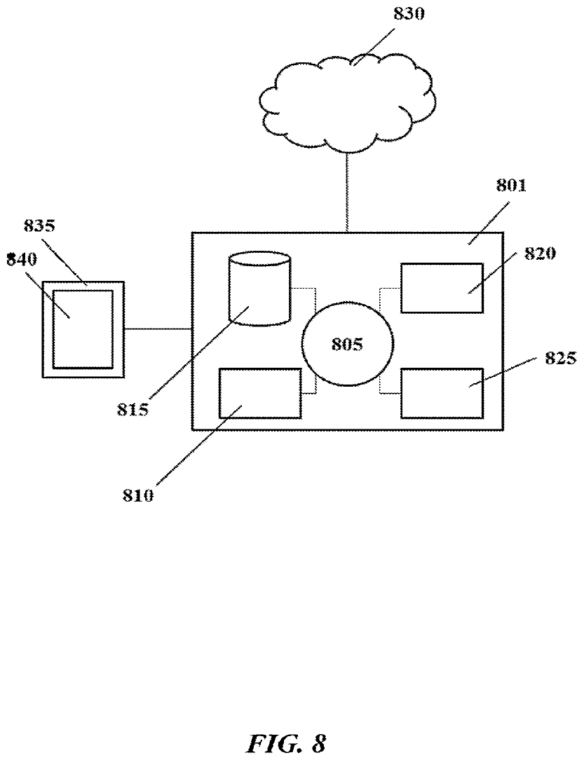

The present disclosure provides computer control systems that are programmed to implement methods of the disclosure. FIG. 8 shows a computer system 801 that is programmed or otherwise configured to control or regulate the microfluidic system. The computer system 801 can regulate various aspects of the microfluidic system of the present disclosure, such as, for example, the stepwise flow of various fluids through channel segments of a channel network, and/or use negative or positive pressure to subject a fluid to flow through a channel network. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., technician or researcher). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a sample readout, such as results upon assaying a biological sample, or instructions for using systems of the present disclosure to process a biological sample(s). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for controlling filling of a microfluidic network comprising:
providing a microfluidic channel network comprising a first channel segment and a second channel segment intersecting the first channel segment at a first junction;
providing a first fluid in the first channel segment up to the first junction, wherein capillary flow of the first fluid is interrupted at the first junction;
providing a second fluid in the second channel segment up to the first junction, wherein capillary flow of the second fluid is interrupted at the first junction; and
providing pressure to both the first channel segment and the second channel segment to control filling of the microfluidic channel network by releasing the interrupted flow of the first fluid and the second fluid into the microfluidic channel network.

2. The method of claim 1, wherein the first channel segment comprises a first curved pinning point and the second channel segment comprises a second curved pinning point where the first channel segment and the second channel segment meet the first junction.

3. The method of claim 2, wherein the first curved pinning point and the second curved pinning point are configured and arranged to provide the interruption of capillary flow of the first fluid and the second fluid.

4. The method of claim 3, wherein the first curved pinning point provides a first meniscus of the first fluid against air, and wherein the second curved pinning point provides a second meniscus of the second fluid against air.

5. The method of claim 2, wherein the first curved pinning point and the second curved pinning point each comprise a step feature.

6. The method of claim 5, wherein the step features are configured and arranged to provide a smaller depth at the first junction compared to a depth of the first channel segment and the second channel segment.

7. The method of claim 1, wherein the microfluidic channel network further comprises a third channel segment at the first junction.

8. The method of claim 7, wherein the microfluidic channel network further comprises one or more additional channel segments intersecting the third channel segment at a second junction.

9. The method of claim 8, wherein the released flow of the first and second fluids is interrupted at the second junction.

10. The method of claim 1, further comprising a channel expansion feature arranged and configured to control the rate of flow of the first fluid.

11. The method of claim 10, wherein the rate of flow of the first fluid is reduced.

12. The method of claim 10, wherein the rate of flow of the first fluid is increased.

13. The method of claim 1, wherein the step of providing pressure to both the first channel segment and the second channel segment to control filling of the microfluidic channel network is provided by one or more pumps.

14. The method of claim 1, wherein the step of providing pressure to both the first channel segment and the second channel segment to control filling of the microfluidic channel network is provided by one or more compressors.

15. The method of claim 1, further comprising regulating the pressure to both the first channel segment and the second channel segment by way of a computer system, thereby regulating filling of the microfluidic channel network.

16. The method of claim 1, wherein the step of providing pressure to both the first channel segment and the second channel segment to control filling of the microfluidic channel network facilitates droplet generation in the microfluidic channel network.

17. The method of claim 1, wherein the first fluid and the second fluid are different.

18. The method of claim 1, wherein the first fluid and the second fluid are the same.

19. The method of claim 1, wherein the first fluid is an aqueous fluid.

20. The method of claim 1, wherein the second fluid is an aqueous fluid.
